(19) United States
(12) Patent Application Publication
(10) Pub. No.: US 2011/0172513 A1
(43) Pub. Date: Jul. 14, 2011

(54) BIOLOGICAL INFORMATION IMAGING APPARATUS

(75) Inventors: Takao Nakajima, Kawasaki-shi (JP); Kazuhiko Fukutani, Yokohama-shi (JP)

(73) Assignee: CANON KABUSHIKI KAISHA, Tokyo (JP)

(21) Appl. No.: 13/063,703

(22) PCT Filed: Nov. 9, 2009
(86) PCT No.: PCT/JP2009/066322

§ 371 Date: Mar. 11, 2011

(30) Foreign Application Priority Data
Sep. 12, 2008 (JP) 2008235543
Sep. 9, 2009 (JP) 2009-208506

(51) Int. Cl. A61B 5/05

(52) U.S. Cl. 600/407

(57) ABSTRACT

The biological information imaging apparatus includes an acoustic wave detector 107 that detects an acoustic wave that is generated from a light absorber 105 and converts it to a first electrical signal; a photo-detector 110 that detects intensities of the light corresponding to a plurality of propagation distances of the light which propagates through the specimen 110 and converts it to a second electrical signal; a signal processing apparatus 111 that derives an average effective attenuation coefficient μ_{eff} of the specimen 110 based on the second electrical signal and derives an optical absorption coefficient μₐ of the specimen 110 based on the first electrical signal and the average effective attenuation coefficient μ_{eff}; and an image constructing apparatus 111 that constructs an image of the distribution of the optical absorption coefficient μₐ based on the distribution of the optical absorption coefficient μₐ.

### TECHNICAL FIELD

The present invention relates to a biological information imaging apparatus.

### BACKGROUND ART

Recently, biological information imaging apparatuses for acquiring an image from information of a living body by using technologies such as X-rays, ultrasonic waves, and Magnetic Resonance Imaging (MRI) technologies have been widely used in a medical field. In addition, optical imaging apparatuses where a light irradiated from a light source such as a laser is allowed to propagate through a living body or the like and the propagated light and the like is detected so as to obtain information on the living body have been actively researched in the medial field. As one of the optical image technologies, a photoacoustic tomography (PAT) is proposed (for example, referred to Non-Patent Document 1).

In a photoacoustic tomography, a pulse light generated from a light source is irradiated to a living body that is a specimen, and an acoustic wave generated from a biological tissue that absorbs energy of the light propagating and diffusing through the living body is detected at a plurality of positions. In the specification, the acoustic wave is sometimes referred to as a “photoacoustic wave”. Next, the signal is analyzed, so that information of optical property values of the living body is displayed as an image. Therefore, the information of the optical property distribution of the living body, particularly, an optical energy absorption density distribution can be acquired in an easily visible form.

According to Non-Patent Document 1, in the photoacoustic tomography, an initial sound pressure P₀ of a photoacoustic wave generated from a light absorber located at a specific position in the specimen due to light absorption can be expressed by the following formula (1).

[Formula 1]

P₀=Γ·μₐ·Φ (1)

Herein, Γ is a Grüneisen coefficient obtained by dividing a product of a thermal expansivity β and a square of a speed of sound c by a specific heat at constant pressure C_{P}. In addition, μₐ is an optical absorption coefficient of the light absorber, and Φ is a light amount in a local region (a light amount irradiated to the light absorber located in a specific position; sometimes, referred to as light fluence). Since the parameter Γ is known to be substantially constant according to the tissues of the living body, the distribution of the product of the optical absorption coefficient βₐ and the light amount Φ, that is, optical energy absorption density distribution of the specimen can be obtained by measuring and analyzing a time change of the sound pressure P that is a magnitude of the acoustic wave at a plurality of the positions.

### CITATION LIST

### Non-Patent Literature

Non-Patent Literature 1: M, Xu, L. V. Wang “Photoacoustic imaging in biomedicine”, Review of scientific instruments, 77, 041101 (2006)

In the above conventional photoacoustic tomography, as understood from the formula (1), the distribution of the optical absorption coefficient μₐ of the specimen cannot be obtained by acquiring only the optical energy absorption density distribution through the measurement of the time change in the sound pressure P. In other words, the distribution of light amount Φ irradiated to the light absorber that generates the photoacoustic wave as well as the optical energy absorption density distribution needs to be obtained in some way.

The light irradiated to the living body is attenuated through the living body. Under the assumption that a light amount Φ₀ irradiated by a light source is constant and the light is irradiated to a larger region than a propagation length of the light in the living body so that the light propagates through the living body like a plane wave, the distribution of light amount Φ of the living body can be approximated to the following formula (2).

[Formula 2]

Φ=Φ₀·exp(−μ_{eff}·d₁) (2)

Herein, μ_{eff} is an average effective attenuation coefficient in the living body. The term “average effective attenuation coefficient” means the “effective attenuation coefficient under the assumption that optical properties are spatially uniform in the living body”. In addition, d₁ is a distance (that is, a depth) from the region (light irradiated region) of the living body which a light is irradiated from a light source to the light absorber in the living body.

In this case, the initial sound pressure P₁ of the generated photoacoustic wave can be expressed by the following formula (3) based on the formula (1).

[Formula 3]

P₁=Γ·μₐ·Φ=Γ·μₐ·Φ₀·exp(−μ_{eff}·d₁) (3)

Therefore, the distribution of the optical absorption coefficient μₐ of the specimen can be acquired by obtaining the average effective attenuation coefficient μ_{eff}. Although the average effective attenuation coefficients μ_{eff} of the living body are already known with regard to some portions, the effective attenuation coefficients μ_{eff} are different among persons. In addition, the distribution of light amount Φ is exponentially changed with respect to the average effective attenuation coefficient μ_{eff} as expressed in the formula (2). Therefore, if the average effective attenuation coefficient μ_{eff} is different, the distribution of light amount Φ becomes greatly different. If there is an error in the distribution of light amount Φ, the distribution of the optical absorption coefficient μₐ of the specimen obtained as the result is also greatly different from the correct value. Therefore, there is a need to measure the average effective attenuation coefficient μ_{eff} of each person. In addition, as optical coefficients of the living body, there are an optical absorption coefficient μₐ, an equivalent scattering coefficient μₛ′, an effective attenuation coefficient μ_{eff}, and the like, and the following formula (4) is satisfied therebetween.

[Formula 4]

μ_{eff}=\sqrt{3μₐ·(μₛ′+μₐ)} (4)

### SUMMARY OF INVENTION

The present invention has been made in view of the above issues and an object thereof is to provide an imaging apparatus for a biological image using a photoacoustic tomography capable of more accurately acquiring a distribution of an optical absorption coefficient μₐ of a specimen by obtaining an average effective attenuation coefficient μ_{eff} unique to the living body that is the specimen in advance.

In order to solve the above problems, there is provided an imaging apparatus having the following configuration. That is, the biological information imaging apparatus of the invention comprising:

a light source unit having a single light source or a plurality of light sources;

an acoustic wave detector that detects an acoustic wave generated from a light absorber in a living body which absorbs a portion of energy of a light irradiated to the living body by the light source unit and converts the acoustic wave to a first electrical signal;

a photo-detector that detects intensities of the light corresponding to a plurality of propagation distances of the light irradiated to the living body by the light source unit and propagates through the living body and converts the intensities of the light to a second electrical signal;

a signal processing apparatus that derives an average effective attenuation coefficient of the living body based on the second electrical signal and derives an optical property distribution of the living body based on the first electrical signal and the average effective attenuation coefficient; and

an image constructing apparatus that constructs an optical property distribution image of the living body based on the optical property distribution of the living body derived by the signal processing apparatus.

According to the biological information imaging apparatus of the invention, it is possible to more accurately obtain the average effective attenuation coefficient μ_{eff} unique to the living body that is the specimen and to more accurately acquire the distribution of the optical absorption coefficient μₐ of the living body.

Further features of the present invention will become apparent from the following description of exemplary embodiments with reference to the attached drawings.

### BRIEF DESCRIPTION OF DRAWINGS

FIG. 1 is a schematic view illustrating a configuration of a biological information imaging apparatus according to a first embodiment of the invention.

FIGS. 2A and 2B is a view for explaining a configuration of changing a distance between a light irradiated position and a photo-detector according to an embodiment of the invention.

FIG. 3 is a view illustrating an example of a light propagation model used to analytically calculate a light amount Φ(ρ) according to an embodiment of the invention.

FIG. 4 is a view illustrating comparison of the light amount Φ(ρ) analytically calculated using the light propagation model according to the embodiment of the invention with a light amount Φ(ρ) calculated using a finite element method.

FIG. 5 is a view for explaining fitting of a graph of the light amount Φ(ρ) analytically calculated according to the invention to a light amount detected by a photo-detector.

FIG. 6 is a flowchart of processes performed by the biological information imaging apparatus according to the first embodiment of the invention.

FIG. 7 is a schematic view illustrating a configuration of a biological information imaging apparatus according to a second embodiment of the invention.

### DESCRIPTION OF EMBODIMENTS

Preferred embodiments of the present invention will now be described in detail in accordance with the accompanying drawings.

### First Embodiment

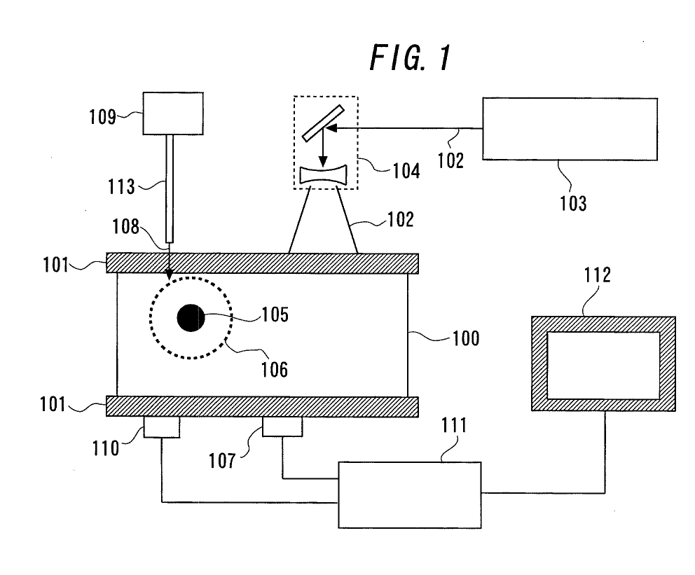

FIG. 1 illustrates a biological information imaging apparatus according to a first embodiment of the invention. The biological information imaging apparatus described in the embodiment is an apparatus that can display an optical property distribution of a living body and a concentration distribution of substances constituting a biological tissue obtained from the information as an image in order to diagnose a malignant tumor or a disease in a blood vessel or observe progress of chemical treatment.

In the biological information imaging apparatus according to the embodiment, a specimen 100 that is a living body is interposed and fixed between two fixing members 101. In addition, a first light 102 irradiated from a first light source 103 is guided to the specimen 100 through an optical unit 104 constructed with lens and the like to be irradiated to the specimen 100. At this time, energy of the first light 102 is absorbed by a light absorber 105 such as a blood vessel, so that an acoustic wave 106 is generated. The acoustic wave 106 is detected by an acoustic wave detector 107 and converted to a first electrical signal.

On the other hand, a second light 108 emitted from a second light source 109 is irradiated to the specimen 100 through a light waveguide 113. The second light 108 that propagates the specimen 100 to be emitted from the specimen 100 is detected by a photo-detector 110 that is disposed to face an irradiated portion of the second light 108 with the specimen 100 interposed therebetween and converted to a second electrical signal. The first electrical signal and the second electrical signal are analyzed by a signal processing unit 111, so that an optical property distribution of the specimen 100 is calculated from the signals. In the signal processing unit 111, image data representing the calculated optical property distribution are constructed. A display apparatus 112 displays the optical property distribution as an image by using the image data. In addition, the fixing members 101 are configured to transmit the first light 102 and the second light 108. In other words, the fixing members 101 may be made of a material of transmitting the first light 102 and the second light 108. In addition, the fixing member 101 may be configured so that the specimen 100 is exposed at the irradiated position.

Herein, an initial sound pressure of the acoustic wave is expressed by the formula (1) as described above. Therefore, under the assumption that the Grüneisen coefficient Γ is a constant value in tissues of the living body, a generation distribution of the initial sound pressure can be obtained by measuring and analyzing a time change of the sound pressure P detected at a plurality of the positions by the acoustic wave detector 107. In addition, the distribution of the product of the optical absorption coefficient μₐ and the light amount Φ (optical energy absorption density distribution) can also be obtained. However, only the distribution of the product of the optical absorption coefficient μₐ and the light amount Φ (optical energy absorption density distribution) can be obtained from the first electrical signal obtained by the acoustic wave detector 107. Therefore, in order to obtain the distribution of the optical absorption coefficient μₐ of the specimen, the optical energy absorption density distribution needs to be corrected with the light amount Φ.

On the other hand, in the case where the light is irradiated to a much larger region than a propagation length of the light in the specimen 100, the light amount Φ can be expressed by the following formula (2). Accordingly, since the light amount Φ can be obtained by obtaining the average effective attenuation coefficient μ_{eff} of the specimen 100, distribution of the optical absorption coefficient μₐ of the specimen 100 can be obtained.

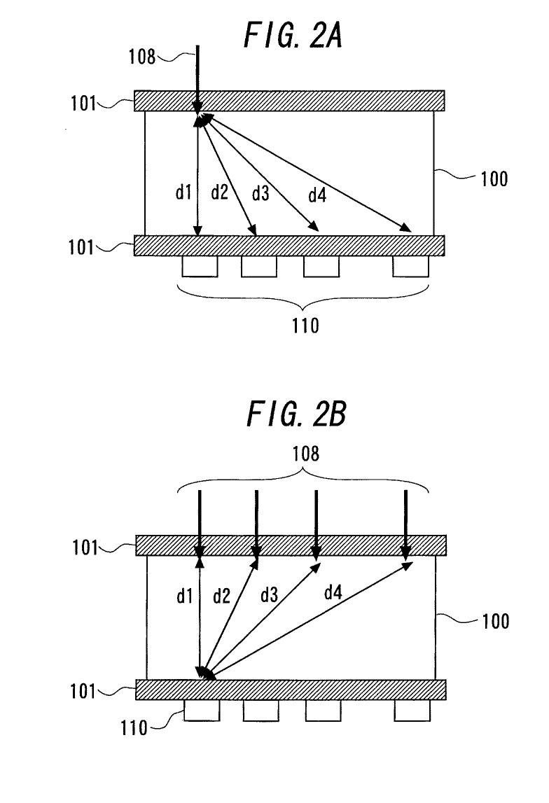

In the embodiment, the second electrical signal obtained by detecting the second light 108 is used so as to obtain the average effective attenuation coefficient μ_{eff}. Herein, the photo-detector 110 scans the fixing member 101, so that the second light can be detected at a plurality of the positions. On the other hand, the second light 108 that is irradiated from the second light source 109 is irradiated to a predetermined position in a spot shape through the light waveguide 113. At this time, as shown in FIG. 2(a), by scanning the photo-detector 110, the distance between the irradiated positions of the second light 108 and the photo-detector 110 can be changed. The distance between the light irradiated position and the photo-detector is referred to as a “propagation distance of light”.

In addition, the light detection is performed at a plurality of the positions, and the detected light amount is plotted according to the distance. The average effective attenuation coefficient μ_{eff} can be obtained by performing fitting to the plotted result, using the theoretical formula expressing the distribution of the optical amount distribution in the specimen 100, which depends on the shape of the specimen 100 (this is, the theoretical formula of the distribution of the intensity (in the specimen 100) of the light irradiated to the specimen 100 and propagates through the specimen 100). Although the photo-detector 110 is scanned so as to change the distance between the irradiated position of the second light 108 and the photo-detector 110 in the embodiment, as shown in FIG. 2(b), the photo-detector 110 may be fixed and the irradiated position of the second light 108 may be scanned by using an optical fiber so as to change the distance. In other words, it is preferable that a plurality of light beams corresponding to the different distance (propagation distances of the light beams) from the light irradiated positions to the photo-detector 110 can be measured. In addition, in the embodiment, the configuration where the irradiated portions of the second light 108 and the photo-detector 110 are disposed to face each other with the specimen 100 interposed therebetween also denotes the positional relationship shown in FIGS. 2(a) and 2(b).

As a result, the average effective attenuation coefficient μ_{eff} of the living body is obtained, and the light amount Φ is obtained by using the obtained average effective attenuation coefficient μ_{eff}. Next, the distribution of the optical absorption coefficient μₐ of the specimen can be obtained by correcting the distribution of the product of the optical absorption coefficient μₐ and the light amount Φ (optical energy absorption density distribution) obtained from the first electrical signal with the obtained light amount Φ. More specifically, the value of the optical energy absorption density may be divided by the light amount at each local position of the specimen.

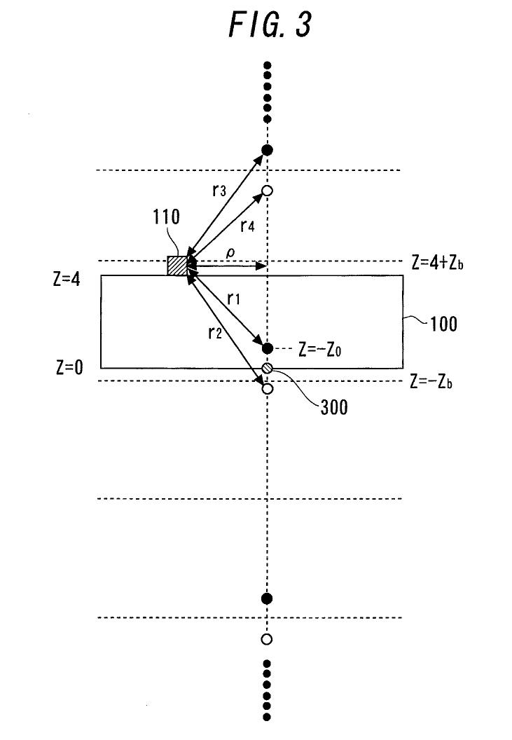

Next, an example of a fitting model is described with reference to FIG. 3. Herein, a case where a measurement position of the specimen 100 (a portion of an object of measurement) has a shape of a parallel flat plate (shape of a slab) is taken into consideration (this case corresponds to fitting the shape of the measurement position (portion of the object of measurement) of the living body to a predetermined model shape to which the above theoretical formula can be adapted in the embodiment). Light propagation in a medium having strong scattering such as a living body can be expressed by a light diffusion equation. The light diffusion equation can be solved analytically with respect to a simple shape such as an infinite parallel flat plate.

If the specimen in FIG. 3 is approximated to an infinite flat plate, the light amount Φ(ρ) that the photo-detector 110 detects from the light irradiated from the light irradiated position 300 can be expressed by the following formula (5) under the assumption that there are an infinite number of dipolar (positive-negative) pseudo light sources (Reference Document: M. S. Patterson et. al. “Time resolved reflectance and transmittance for the noninvasive measurement of tissue optical properties”, Applied Optics, 28, 2331 (1989))

$$\begin{matrix}[ Formula 5 ] & \\ φ ( ρ ) = C \cdot ( \frac{{e}^{- {μ}_{eff} {r}_{1}}}{{r}_{1}} - \frac{{e}^{- {μ}_{eff} {r}_{2}}}{{r}_{2}} + \frac{{e}^{- {μ}_{eff} {r}_{3}}}{{r}_{3}} - \frac{{e}^{- {μ}_{eff} {r}_{4}}}{{r}_{4}} + … ) & ( 5 )\end{matrix}$$

Herein, μ is a distance from the point that faces the irradiated position 300 with the specimen 100 interposed therebetween to the photo-detector 110, and C is a coefficient depending on diffusion. In addition, rᵢ is a distance between an i-th pseudo light source and the photo-detector 110 and is a function of ρ and a diffusion coefficient. For the approximation, the diffusion coefficient is set to an integer number.

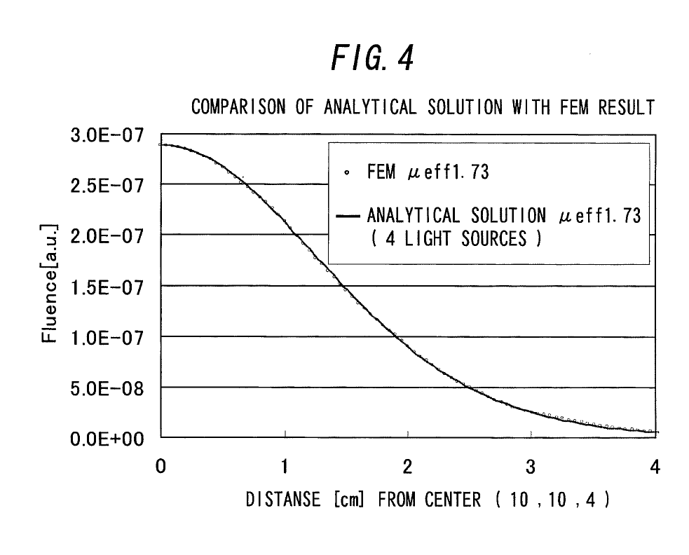

FIG. 4 illustrates a comparison of the distribution of light amount Φ(ρ) derived by using the formula (5) with the distribution of light amount Φ(ρ) derived by solving a light diffusion equation according to a finite element method. It can be understood from substantial coincidence of the two results that the distribution of light amount Φ(ρ) of the specimen 100 having a shape of a parallel flat plate (shape of a slab) can be expressed by the model of the formula (5).

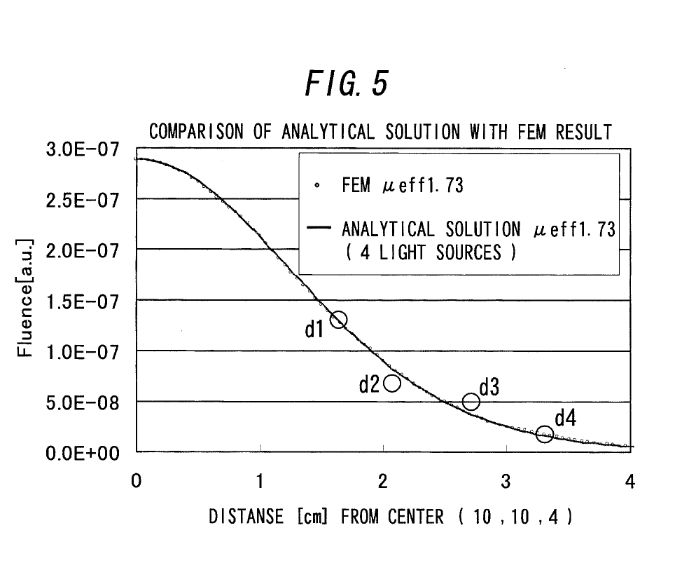

Therefore, the light amount is detected by changing the ρ, and as shown in FIG. 5, the resulting measurement is fitted by using the formula (5), so that the average effective attenuation coefficient μ_{eff} of the specimen can be obtained.

Next, the embodiment is described in detail. In FIG. 1, the first light source 103 and the second light source 109 irradiate the light having the wavelength absorbed by specific substances constituting the living body that is the specimen 100. In addition, the first light source 103 and the second light source 109 irradiate the light having the same wavelength. In addition, the first light source 103 is a light source for generating the photoacoustic wave, which includes at least one pulse light source that can generate a pulse light having a pulse width in the order of several nano seconds to hundreds of nano seconds. A laser is preferably used as the first light source 103. However, instead of the laser, a photodiode or the like may be used. As the laser, a solid state laser, a gas laser, a dye laser, a semiconductor laser, and other various lasers may be used.

In addition, although the number of the first light source 103 is one in the embodiment, a plurality of the light sources may be used. In this case, in order to increase the intensity of the light irradiated to the living body, a plurality of the light sources oscillating at the same wavelength may be used. In addition, in order to measure a difference in wavelength in the optical property distribution, a plurality of the light sources having different oscillation wavelengths may be used. In addition, if a dye laser, an optical parametric oscillator (OPO) or a titan sapphire laser, of which the oscillating wave length is convertible, can be used as the light source 103, the difference in wavelength in the optical property distribution can be measured. It is preferable that the wavelength of the first light source 103 is in a range of 700 nm to 1100 nm, where the absorbance is low in the living body. In addition, in the case where the optical property distribution of the biological tissue relatively in the vicinity of the surface of the living body is obtained, the wavelength range wider than the above wavelength range, for example, a range from 400 nm or more to 1600 nm or less may be used. The wavelength range of the second light source 109 may be the same as the above wavelength range.

The second light source is used to irradiate the light that is to be detected by the photo-detector 110. It is preferable that the second light source 109 is a light source that can generate an intensity-modulated light. The second light source 109 may generate a continuous light having a waveform different from that of the pulse light. In addition, the second light source 109 may generate a pulse light similarly to the first light source 103. More specifically, a laser is preferably used. However, instead of the laser, a photodiode or the like may be used. As an example of the laser, a semiconductor laser is preferable. However, a gas laser, a dye laser, a solid state laser, and other various lasers may be used.

The first light 102 irradiated from the first light source 103 may be irradiated to the specimen by using only the optical unit 104 or be propagated by using the light waveguide or the like. It is preferable that the light waveguide is an optical fiber. In the case where the optical fiber is used, a plurality of the optical fibers may be used for a plurality of the light sources so as to guide the light to the surface of the living body. In addition, the light beams from a plurality of the light sources may be introduced to a single optical fiber, so that all the light beams can be guided to the living body by using only one optical fiber. The optical unit 104 shown in FIG. 1 is constructed with general optical parts such as mirrors and lenses. The optical unit 104 has a function of changing the direction of the first light 102 emitted from the first light source 103 or functions of condensing, magnifying, and shaping the first light 102. The optical parts constituting the optical unit 104 may be any combination that can allow the first light 102 to be irradiated to the specimen 100 in a desired shape and area.

It is preferable that the light waveguide 113 that guides the second light 108 from the second light source 109 into the living body is an optical fiber. In addition, it is preferable that the second light 108 is irradiated to the specimen 100 in a spot shape. The light absorber 105 in the specimen 100 is a portion having a high optical absorption coefficient in the specimen 100. For example, if a human body is the object of measurement, the light absorber may be hemoglobin, a blood vessel containing a large amount of hemoglobin, or a malignant tumor. The acoustic wave detector (or probe) 107 detects the acoustic wave 106 generated from the light absorber 105 absorbing a portion of energy of the first light 102 propagating through the living body and converts the acoustic wave to the first electrical signal. The acoustic wave detector 107 may be any sound wave detector that can detect the acoustic wave signal such as a transducer using a piezo-electric phenomenon, a transducer using a resonance of light, and a transducer using a change of capacitance. In addition, an array of the transducers may be used, and a single transducer may be used.

In addition, in the embodiment, in order to detect the acoustic wave 106 at a plurality of positions, the surface of the fixing member 101 is two-dimensionally scanned by a single acoustic wave detector 107, so that the acoustic wave 106 can be detected at a plurality of the positions. Alternatively, if the acoustic wave 106 can be detected at a plurality of the positions, the same effect can be obtained. Therefore, a plurality of the acoustic wave detectors may be disposed on the surface of the fixing member 101. In addition, it is preferable that an acoustic impedance matching material such as gel or water is interposed between the acoustic wave detector 107 and the fixing member 101 so as to suppress the reflection of the acoustic wave 106.

The photo-detector 110 detects the second light 108 that propagate and transmit through the specimen (living body) 100 and converts the second light 108 to the second electrical signal. The photo-detector 110 may be any optical detector capable of detecting light such as a photodiode, an avalanche photodiode, a photomultiplier tube, and CCD. In addition, in the embodiment, in order to change the distance between the irradiated position of the second light 108 and the photo-detector 110 and to detect the light at a plurality of the position, the surface of the fixing member 101 is scanned by a single photo-detector 110. However, if the light can be detected at a plurality of the positions, the same effect can be obtained. As described above, a plurality of the photo-detectors 110 may be disposed on the surface of the fixing member 101.

In addition, if the measurement of detecting the acoustic wave 106 generated due to the irradiation of the first light 102 by the acoustic wave detector 107 is denoted by a first measurement and the measurement of detecting the light due to the irradiation of the second light 108 by the photo-detector 110 is denoted by a second measurement, it is preferable that the first measurement and the second measurement are not simultaneously performed. In this case, the first and second measurements may be alternately performed. In addition, after the one of the measurements is completed, the other may be performed.

The signal processing unit 111 analyzes the first electrical signal and the second electrical signal and calculates information on the optical property distribution of the specimen (living body) 100 by using the signals. The signal processing unit 111 calculates the optical property distribution such as the distribution of the optical absorption coefficient μₐ and the optical energy absorption density distribution based on the first electrical signal obtained by the acoustic wave detector 107 and the second electrical signal obtained by the photo-detector 110. In addition, the signal processing unit 111 can calculate the position and size of the light absorber 105 in the specimen (living body) 100. In addition, the signal processing unit 111 may be any unit that can store the first electrical signal and the second electrical signal and converts the electrical signals to the data of the optical property distribution by a calculation unit. For example, an oscilloscope and a computer that can analyze the data stored in the oscilloscope can be used.

In this case, by the program stored in the computer, a calculation unit (CPU) may calculate the first electrical signal and the second electrical signal and convert the signals to the data of optical property distribution. In addition, by the program, the image data that are to be displayed on the display apparatus 112 may be constructed. Alternatively, a separate memory may be provided to the signal processing unit 111, so that the first electrical signal and the second electrical signal are stored in the memory. In addition, by a program separately stored in the memory of the signal processing unit 111, the calculation unit (CPU) may calculate the first electrical signal and the second electrical signal and convert the signals to the data of optical property distribution, so that the image data can be constructed.

The signal processing unit 111 obtains a generating distribution of the initial sound pressure P₀ or a distribution of a product of an optical absorption coefficient μₐ and a light amount Φ (optical energy absorption density distribution) from the first electrical signal. In addition, the signal processing unit 111 obtains average effective attenuation coefficient μ_{eff} from the second electrical signal by using the fitting described above. In addition, the signal processing unit 111 obtains a distribution of optical absorption coefficient μₐ in specimen 100 by correcting the light amount by using the obtained average effective attenuation coefficient μ_{eff} with respect to the distribution of the product of the optical absorption coefficient μₐ and the light amount Φ (optical energy absorption density distribution). In addition, the signal processing unit 111 generates image data that are used to display information such as the generating distribution of the initial sound pressure P₀, the distribution of the product of the optical absorption coefficient μₐ and the light amount Φ (optical energy absorption density distribution), and the distribution of the optical absorption coefficient μₐ on the image display apparatus 112. The image data corresponds to the optical property distribution image of the living body in the embodiment.

The image display apparatus 112 of FIG. 1 may be any apparatus on which the image data generated by the signal processing unit 111 can be displayed. For example, a liquid display can be employed. In addition, in the case where a plurality of light having different wavelengths are used, the distribution of the optical absorption coefficient μₐ of the specimen 100 may be calculated by the above system with respect to each of the wavelengths. Therefore, a concentration distribution of substances constituting the living body can be displayed as an image by comparing wavelength dependency specific to the substances (glucose, collagen, oxidized/reduced hemoglobin, and the like) constituting the biological tissue with the distribution of the optical absorption coefficient μₐ at each wavelength.

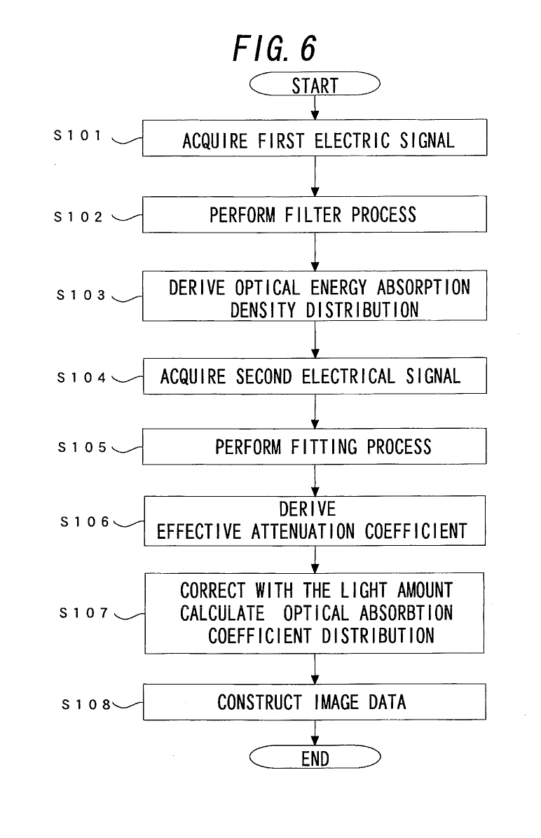

FIG. 6 illustrates a flowchart of processes of the biological information imaging apparatus according to the invention. Steps corresponding to the processes of the signal processing unit 111 in the flowchart are executed by a program stored in the signal processing unit 111. If the flowchart is executed, firstly, in step S101, the first electrical signal is acquired by the acoustic wave detector 107. At this time, the acoustic wave detector 107 detects the acoustic wave at a plurality of positions by scanning the fixing member 101. If the process of step S101 is completed, the procedure proceeds to step S102.

In step S102, a filter process is performed on the first electrical signal obtained in step S101. If the process of step S102 is completed, the procedure proceeds to step S103.

In step S103, optical energy absorption density distribution that is the distribution of the product of the optical absorption coefficient μₐ and the light amount Φ is calculated from the first electrical signal after the filter process. If the process of step S103 is completed, the procedure proceeds to step S104.

In step S104, the second electrical signal is acquired by the photo-detector 110. At this time, the photo-detector 110 detects the light transmitting the specimen 100 at a plurality of positions by scanning the fixing member 101. The detection corresponds to the detection of the intensities of the light propagating through the living body (light emitted from the living body after propagating the living body) corresponding to a plurality of propagation distances of the light. If the process of step S104 is completed, the procedure proceeds to step S105.

In step S105, a fitting process is performed. More specifically, values of parameters are set so that the second electrical signal (a plurality of the values acquired at a plurality of the positions) acquired in step S104 can be fitted to the theoretical formula of the light amount Φ expressed by the formula (5). If the process of step S105 is completed, the procedure proceeds to step S106.

In step S106, the average effective attenuation coefficient μ_{eff} is calculated in the state where the second electrical signal (a plurality of the values acquired at a plurality of the positions) is best fitted to the theoretical formula of the light amount Φ expressed by the formula (5) in step S105. The value becomes the average effective attenuation coefficient μ_{eff} of the specimen (living body) 100 in the measurement. If the process of step S106 is completed, the procedure proceeds to step S107.

In step S107, the light amount Φ is obtained from the average effective attenuation coefficient μ_{eff} calculated in step S106 and the formula (2), and the distribution of the optical absorption coefficient μₐ is calculated by correcting the optical energy absorption density distribution with the light amount Φ. In other words, the distribution of the optical absorption coefficient μₐ is calculated by correcting the distribution of the product of the optical absorption coefficient μₐ and the light amount Φ (optical energy absorption density distribution) with the light amount Φ. If the process of step S107 is completed, the procedure proceeds to step S108.

In step S108, the image data that are to be displayed on the display apparatus 112 are constructed from the optical absorption coefficient μₐ obtained in step S107. If the process of step S108 is completed, the main routine is ended.

The processes for the first electrical signal and the second electrical signal are not necessarily performed in accordance with the order in the flowchart. The processes (S104 to S106) for the second electrical signal may be firstly performed, and after that, the processes (S101 to S103) for the first electrical signal may be performed. Alternatively, the acquisition processes (S101 and S104) for the first and second electrical signals may be firstly performed, and after that, the other processes may be performed.

As described hereinbefore, by using the biological information imaging apparatus according to the embodiment, it is possible to accurately obtain the optical property distribution of the living body, particularly, the distribution of the optical absorption coefficient μₐ and to display the distribution as an image.

In addition, in the embodiment, the light source unit is configured to include a first light source 103, an optical unit 104, a second light source 109, and a light waveguide 113. In addition, in the embodiment, the signal processing unit 111 corresponds to a signal processing apparatus and a image constructing apparatus.

In addition, in the flowchart, the process of step S101 corresponds to the acoustic wave detecting process. In addition, the process of step S103 corresponds to the absorption density distribution calculating process. The process of step S104 corresponds to the light detecting process. The process of step S106 corresponds to the average attenuation coefficient deriving process. The process of step S107 corresponds to the optical property distribution deriving process. The process of step S108 corresponds to the image constructing process. In addition, some of the steps of the flowchart may be executed by a program stored in the signal processing unit 111, and others may be executed manually.

In addition, in the case where the fixing member 101 is made of a material of transmitting the second light as described above, the photo-detector 110 may be fixed to the fixing member 101. In this case, the photo-detector 110 detects the intensity of light in the vicinity of the surface of the living body. On the other hand, in the case where the photo-detector 110 is directly mounted on the specimen 100, the photo-detector 110 detects the intensity of light in the surface of the living body.

In addition, although a blood vessel, a malignant tumor, or the like is exemplified as the light absorber 105 in the embodiment, the light absorber 105 of the invention is not limited thereto. For example, the contrast agent introduced into the living body may be treated as the light absorber 105. In addition, as described above, the effective attenuation coefficient μ_{eff} is calculated as the average optical property value of the living body, and the distribution of the optical absorption coefficient μₐ is obtained by using the value. Besides the effective attenuation coefficient μ_{eff}, by taking into consideration a relationship between the optical absorption coefficient μₐ and a scattering coefficient μₛ, an equivalent scattering coefficient μₛ′, and the like, the distribution of the optical absorption coefficient μₐ may be obtained by using the values of the scattering coefficient μₛ and the equivalent scattering coefficient μₛ′.

### Second Embodiment

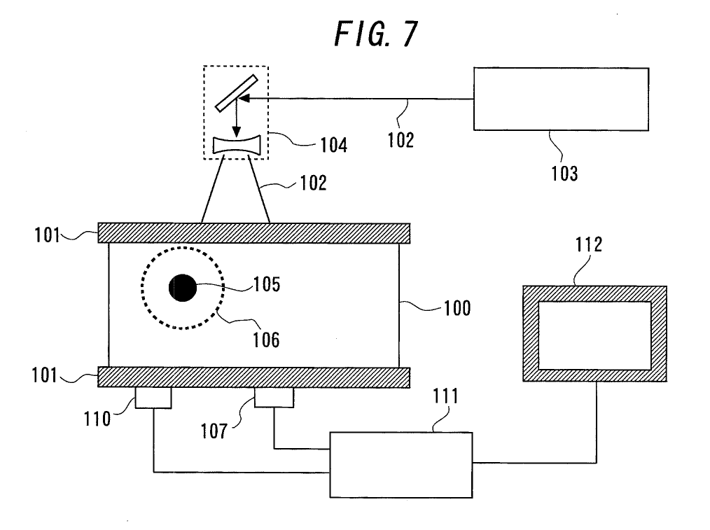

Now, a second embodiment of the invention will be described with reference to the drawings. The embodiment is an example where the second electrical signal is also detected by the first light 102 so as to obtain the effective attenuation coefficient μ_{eff}. FIG. 7 illustrates a biological information imaging apparatus according to the embodiment. The biological information imaging apparatus according to the embodiment is different from that of the first embodiment in that the second light source 109 and the light waveguide 113 are not included and the second light 108 is not used. Hereinafter, the same components as the first embodiment are denoted by the same reference numerals, and the description thereof will not be repeated. Only the features different from those of the first embodiment is described.

In the embodiment, the second electrical signal that is obtained by detecting the first light 102, which is irradiated from the first light source 103 and transmits the specimen (living body) 100, by the photo-detector 110 is used so as to obtain the average effective attenuation coefficient μ_{eff} of the specimen. In addition, the light detection is performed at a plurality of positions, and the detected light amount is plotted according to the distance between the light irradiated positions and the photo-detector 110. Similarly to the first embodiment, the average effective attenuation coefficient μ_{eff} is obtained by performing fitting to the plotted result, using the theoretical formula depending the shape of the specimen (living body) 100.

The distribution of the optical absorption coefficient μₐ of the specimen can be obtained by light-amount-correcting the distribution of the product of the optical absorption coefficient μₐ and the light amount Φ (optical energy absorption density distribution) obtained from the first electrical signal by using average effective attenuation coefficient μ_{eff}. Similarly to the first embodiment, the acoustic wave detector 107 detects the acoustic wave 106 generated from the light absorber 105 that absorbs a portion of energy of the light 102 propagating through the specimen (living body) 100 and converts the acoustic wave to the first electrical signal.

In addition, if the measurement of detecting the acoustic wave 106 generated due to the irradiation of the light 102 by the acoustic wave detector 107 is denoted by a first measurement and the measurement of detecting the light due to the irradiation of the light 102 by the photo-detector 110 is denoted by a second measurement, the first measurement and the second measurement may be simultaneously performed in the embodiment. Alternatively, the first and second measurements may be alternately performed. In addition, after the one of the measurements is completed, the other may be performed. The processes for the obtained first and second electrical signals and other components are the same as those of the first embodiment.

As described above, in the biological information imaging apparatus according to the embodiment, the first electrical signal and the second electrical signal are obtained by using the first light 102 emitted from the first light source 103. In other words, the distribution of the product of the optical absorption coefficient μₐ and the light amount Φ (optical energy absorption density distribution) and the average effective attenuation coefficient μ_{eff} of the living body can be obtained by using only the first light source 103.

Accordingly, the configuration of the apparatus can be simplified, so that it is possible to promote cost reduction. In addition, the first measurement and the second measurement can be simultaneously performed, so that it is possible to increase a degree of freedom of the measurement timings. In addition, the light source unit includes the first light source 103 and the optical unit 104 according to the embodiment, and the embodiment corresponds to the case where the light source unit includes a single light source.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2008-235543, filed on Sep. 12, 2008, and Japanese Patent Application No. 2009-208506, filed on Sep. 9, 2009, which are hereby incorporated by reference herein in their entirety.

### REFERENCE SIGNS LIST

100 specimen (living body)
101 fixing member
102 first light
103 first light source
104 optical unit
105 light absorber
106 acoustic wave
107 acoustic wave detector
108 second light
109 second light source
110 photo-detector
111 signal processing unit
112 display apparatus
113 light waveguide
300 light irradiated position

1. A biological information imaging apparatus comprising:
an acoustic wave detector that detects an acoustic wave generated from a light absorber in a living body which absorbs a light irradiated to the living body and converts the acoustic wave to a first electrical signal;
a photo-detector that detects intensities of the light corresponding to a plurality of propagation distances of the light irradiated to the living body and propagates through the living body and converts the intensities of the light to a second electrical signal;
a signal processing apparatus that derives an average effective attenuation coefficient of the living body based on the second electrical signal and derives an optical property distribution of the living body based on the first electrical signal and the average effective attenuation coefficient; and
an image constructing apparatus that constructs an optical property distribution image of the living body based on the optical property distribution of the living body derived by the signal processing apparatus.

2. A biological information imaging apparatus according to claim 1, wherein the average effective attenuation coefficient is derived by fitting the second electrical signal to a theoretical formula of an intensity distribution of the light in the living body, which is irradiated to the living body to propagate through the living body.

3. A biological information imaging apparatus according to claim 1, wherein the optical property distribution is an optical absorption coefficient distribution of the living body.

4. A biological information imaging apparatus according to claim 2, wherein a shape of a portion of an object of measurement in the living body is fitted to a predetermined model shape to which the theoretical formula can be adapted.

5. A biological information imaging apparatus according to claim 1, wherein an irradiated position of the light and the photo-detector are disposed to face each other with the portion of the object of measurement in the living body interposed therebetween.

6. A biological information imaging apparatus according to claim 1, further comprising a light source unit having a single light source or a plurality of light sources, wherein at least light source that generates the acoustic wave that is to be converted to the first electrical signal by the acoustic wave detector among the light sources of the light source unit is a light source that generates a pulse light.

7. A biological information imaging apparatus according to claim 1, further comprising a light source unit having a plurality of light sources,
wherein the light source unit includes at least two light sources including a first light source that generates a pulse light and a second light source that generates a light having a waveform different from that of the pulse light,
wherein the acoustic wave detector is configured to convert the acoustic wave generated due to the light irradiated to the living body by the first light source to the first electrical signal, and
wherein the photo-detector is configured to convert a light irradiated to the living body by the second light source and propagates through the living body to the second electrical signal.

8. A biological information imaging apparatus according to claim 7, wherein the first light source and the second light source generate a light having the same wavelength.

9. A biological information imaging apparatus according to claim 1, wherein the photo-detector is configured to detect the intensities of the light at a plurality of the positions in the surface of the living body or in the vicinity of the surface of the living body, so that the intensities of the light corresponding to a plurality of the propagation distances of the light that is irradiated to the living body and propagates through the living body can be detected.

10. A biological information imaging apparatus according to claim 1, wherein the acoustic wave detector is configured to detect the acoustic wave at a plurality of positions in the surface of the living body or in the vicinity of the surface of the living body.

11. A biological information imaging apparatus according to claim 1, wherein the light absorber is a contrast agent that is introduced into the living body.

12. A biological information imaging method comprising:
an acoustic wave detecting process of detecting an acoustic wave generated from a light absorber in a living body which absorbs a portion of energy of a light irradiated to the living body and converting the acoustic wave to a first electrical signal;
a light detecting process of detecting intensities of the light corresponding to a plurality of propagation distances of the light irradiated to the living body and propagates through the living body and converting the intensities of the light to a second electrical signal;
an absorption density distribution calculating process of calculating an optical energy absorption density distribution that is a distribution of a product of an optical absorption coefficient of the living body and a light amount based on the first electrical signal;
an attenuation coefficient deriving process of deriving an average effective attenuation coefficient of the living body based on the second electrical signal;
an optical property distribution deriving process of deriving an optical property distribution of the living body based on the optical energy absorption density distribution calculated in the absorption density distribution calculating process and the average effective attenuation coefficient derived in the attenuation coefficient deriving process; and
an image constructing process of constructing an optical property distribution image of the living body based on the optical property distribution of the living body derived in the optical property distribution deriving process.

13. A biological information imaging method according to claim 12, wherein in the attenuation coefficient deriving process, the average effective attenuation coefficient is derived by fitting the second electrical signal to a theoretical formula of an intensity distribution of the light in the living body, which is irradiated to the living body to propagate through the living body.

14. A biological information imaging method according to claim 12, wherein the optical property distribution is an optical absorption coefficient distribution of the living body.

15. A biological information imaging method according to claim 13, wherein a shape of a portion of an object of measurement in the living body is fitted to a predetermined model shape to which the theoretical formula can be adapted.

16. A biological information imaging method according to claim 12, wherein in the light detecting process, an irradiated position of the light in the living body and a position where intensities of the light propagating through the living body corresponding to a plurality of propagation distances are detected are disposed to face each other with a portion of an object of measurement in the living body interposed therebetween.

17. A biological information imaging method according to claim 12, wherein the light irradiated to the living body in the acoustic wave detecting process is a pulse light.

18. A biological information imaging method according to claim 12,
wherein the light irradiated to the living body in the acoustic wave detecting process is a pulse light, and
wherein the light irradiated to the living body in the light detecting process is a light having a waveform different from that of the pulse light.

19. A biological information imaging method according to claim 18, wherein a wavelength of the light irradiated to the living body in the acoustic wave detecting process and a wavelength of the light irradiated to the living body in the light detecting process are the same.

20. A biological information imaging method according to claim 12, wherein in the light detecting process, the intensities of the light are detected at a plurality of the positions in the surface of the living body or in the vicinity of the surface of the living body, so that the intensities of the light corresponding to a plurality of the propagation distances of the light irradiated to the living body and propagates through the living body can be detected.

21. A biological information imaging method according to claim 12, wherein in the acoustic wave detecting process, the acoustic wave is detected at a plurality of positions in the surface of the living body or in the vicinity of the surface of the living body.

22. A biological information imaging method according to claim 12, wherein the light absorber is a contrast agent introduced into the living body.

23. A program which executes on a computer at least one of the absorption density distribution calculating process, the attenuation coefficient deriving process, the optical property distribution deriving process, and the image constructing process in the biological information imaging method according to claim 12.

24. A biological information imaging apparatus according to claim 1, wherein the acoustic wave detector detects an acoustic wave generated from a light absorber in a living body which absorbs a light irradiated to a larger region than a propagation length of the light in the living body.

25. A biological information imaging method according to claim 12, wherein the light irradiated to the living body in the acoustic wave detecting process is a light irradiated to a larger region than a propagation length of the light in the living body.
